(19) United States
(12) Patent Application Publication
(10) Pub. No.: US 2018/0140813 A1
(43) Pub. Date: May 24, 2018

(54) CAP TO PROVIDE SELECTED HEAT ONLY, VIBRATION ONLY AND COMBINED HEAT PLUS VIBRATION TO STIMULATE THE APPLICATION OF A PAIN RELIEVING MATERIAL

(71) Applicant: Bryan Tapocik, Highland, CA (US)

(72) Inventor: Bryan Tapocik, Highland, CA (US)

(21) Appl. No.: 15/396,679

(22) Filed: Jan. 1, 2017

Related U.S. Application Data
(60) Provisional application No. 62/426,170, filed on Nov. 23, 2016.

(51) Int. Cl. A61M 35/00; A61J 1/14; A61J 1/18; B65D 50/04; A61H 1/00

(52) U.S. Cl. CPC A61M 35/003; A61J 1/1418; A61J 1/18; B65D 50/043; A61M 2205/587; A61J 2200/42; A61M 2205/8206; A61M 2205/36; A61H 1/00

(57) ABSTRACT

An accessory cap which is inserted on a container housing a lubricant with an applicator at or adjacent to the front of the container to provide only vibration without heat or heat alone or vibration with heat to facilitate better absorption of the pain relieving lubricant after it has been applied to a person's skin. Also included is a tamper-proof locking assembly to lock the present invention cap onto a container.

### CROSS-REFERENCE TO RELATED APPLICATION

This patent application claims priority to Provisional Application No. 62/426,170 filed on Nov. 23, 2016, now pending.

### BACKGROUND OF THE INVENTION

### 1. Field of the Invention

The field of the invention relates to the area of containers which dispense pain relieving material such as pain relieving gel or lubricant and the containers which retain such material. An applicator is frequency found on the top or front end of the container. The present invention relates to the field of apparatus which help to cause the pain relieving gel or lubricant to be better absorbed after it has been applied to the area of the body from which the source of pain emanates.

### 2. Description of the Prior Art

The following eight patents and published patent applications are the closest prior art known to the inventor.

1. U.S. Pat. No. 3,752,155 issued to William Blinoff, Jr., et al. on Aug. 14, 1973 for “Apparatus for Heating or Cooling a Surface While Simultaneously Dispensing a Liquid Product Thereon” (hereafter the “Blinoff Patent” 0;

2. U.S. Pat. No. 4,722,326 issued to Max J. Ruderian on Feb. 2, 1988 for “Vibratory Therapeutic Device” (hereafter the “Ruderian Patent”);

3. U.S. Pat. No. 6,911,010 issued to Keith Dirks et al. on Jun. 28, 2005 for “Heated Massager with Massaging Liquid Dispenser” (hereafter the “Dirks Patent”);

4. U.S. Pat. No. 7,699,795 issued to Simon Siu Man Nan on Apr. 20, 2010 for “Massager Device with Liquid Applicator” (hereafter the “Nan Patent”);

5. U.S. Pat. No. 7,883,287 issued to Timothy Thorpe on Feb. 8, 2011 for “Dispenser with Thermal Storage Tip” (hereafter the “Thorpe Patent”);

6. United States Published Patent Application No. 2012/0109041 to Jutta Munz on May 3, 2012 for “Applicator Device for Cosmetic and/or Medical Use” (hereafter the “Munz Published Patent Application”);

7. U.S. Pat. No. 8,921,746 issued to David W. Baarman et al. on Dec. 30, 2014 for “Inductively-Heated Applicator System” (hereafter the “Baarman Patent”);

8. PCT Application No. PCT/US86/00216 to Max J. Ruderian on Aug. 28, 1986 for “Vibratory Therapeutic Applicator” (hereafter the Ruderian PCT Application”).

The Blinoff Patent discloses the concept of having a container which contains pain relieving material in it and essentially what is now known as a rollerball where the content is applied to the surface of the skin or affected area by the pain relieving contents in the container being affixed to the rollerball which then applies it to the skin. This patent also discloses the concept of having a heating element so that heat can be applied at the same time that the pain relieving element is placed upon the skin. Specifically, the patent discloses:
“An apparatus is provided for either heating or cooling the skin surface while simultaneously dispensing a liquid product thereon. The apparatus includes a rotatably mounted mass of thermal energy storage material having a hardened, curved heat transfer exterior to which a film of the liquid product adheres when the mass rotates. The mass and the skin surface engaged thereby have disparate temperatures, thus producing either a hot or cold sensation on the skin surface when the mass is in rolling contact therewith and the product is being dispensed thereon.”

This invention discloses the concept of having a heat applicator to heat up the contents.

The Ruderian Patent discloses the concept of a device which contains both a heating and vibrating element within the device to provide heating and vibration when a lubricant or gel is applied to the skin. This patent discloses the concept of having both the heater and the vibrator in the device but this is a separate device and does not contain the contents of the heat or pain relieving liquid or gel within the container. This is simply a device that embodies the concept of applying heat and/or vibration to the lubricant or gel on the affected area. An examination of the figures shows that the heating and vibration element are in the container itself (which does not contain the substance for providing the pain relieving gel or material).

Reference is made to FIG. 1 and in particular, Column 3 Lines 31 through 49 which disclose the following:
“As shown best in FIG. 1, the improved vibratory therapy device 10 comprises the lightweight housing 14 of illustrative generally cylindrical shape and which is advantageously formed from a lightweight and relatively inexpensive impact-resistant molded plastic or the like. The heater/vibrator assembly 12 is mounted within the housing in any suitable manner and includes a lightweight motor 18 for rotatably driving an output shaft 20. An air flow fan 22 of lightweight molded plastic or the like is securely mounted upon the output shaft 20 for rotation therewith upon appropriate connection of the motor 18 to a source of electrical power. A power cord 24 is shown in FIG. 1 for this purpose and may be appropriately connected to an ac or cc power supply, as required, with an adjustable thermostat 26 being mounted along the power cord 24 to provide a combination on-off switch and to regulate the rotational speed of the motor 18 and fan 22.”

Therefore, the concept of having a heating or vibrating element to help facilitate the application of a pain relieving material absorbed on the skin to help treat the affected area is disclosed in this patent. The material for providing heat or cold relief is not within the container but is applied in a separate manner.

The Dirks Patent discloses:
“A hand-held massager includes a housing having a gripping portion and a body-contacting portion, the housing being configured for emitting a massaging liquid and the body-contacting portion being configured for emitting heat to a target surface. The massager also emits a vibration and the amount of vibration is variable, as is the amount of heat and the amount of emitted massaging liquid. A pump is disposed within the housing for dispensing the massaging liquid, and a removable reservoir is in fluid communication with the pump. The reservoir is pivotably mounted to the massager between an operational position in which the reservoir is inclined relative to the body-contacting portion, and a refill position in which the body-contacting portion is placed in a generally vertical position and the reservoir is in a generally parallel position relative to the body-contacting portion.”

Therefore, this patent discloses the concept of having a container containing pain relieving material in it and material being applied through the device and then having a massager which helps to provide the material being absorbed into the skin.

The Nan Patent discloses:
“A massager device that provides a liquid applicator that dispenses a liquid and provides a massaging effect is disclosed. The massager device comprises a massage head adapted to engage a reservoir for storing a liquid, a liquid dispensing component engaged to the reservoir for applying the liquid, and a vibratory component for vibrating the massager device such that the user may simultaneously apply the liquid and provide a massaging effect.”

The Thorpe Patent discloses a dispenser with a thermal storage tip where the container retains the pain relieving material and is applied through the applicator which provides heat to the heat absorbing material after it is applied to the skin.

The Munz Published Patent Application discloses:
“The invention relates to an applicator device, comprising an applicator element (9) for applying a substance (27) present in a substance chamber (25, 27) to a skin section. The applicator device (1) comprises an applicator part (2) having an upper housing part (8) and a vibration part (3) having a lower housing part (4), wherein in the lower housing part (4) a vibration element (10) is provided which can be actuated by a power source (6) and the vibrations of which can be transmitted to the upper housing part (8) and the applicator element (9), and wherein an activator device (22) for generating heating or cooling energy is provided in the upper housing part (8), wherein the heating or cooling energy can be transferred to the substance (27) present in the substance chamber (25, 27).”

The Ruderian PCT Application corresponds to the previously discussed Ruderian Patent which issued as U.S. Pat. No. 4,722,326.

### SUMMARY OF THE INVENTION

The present invention is an accessory cap which is inserted on a container housing a lubricant with an applicator at or adjacent to the front of the container to provide only vibration without heat or heat alone or vibration with heat to facilitate better absorption of the lubricant after it has been applied to a person's skin. In this patent application, pain relieving material includes material selected from the group consisting of oil-based pain reliever, gel-based pain relievers, paste pain relievers, lubricating ointment pain relievers, and selected combinations of lubricants, gels, ointments and pastes used to relieve pain (severally and jointly hereafter referred to as “pain relieving lubricants”). Pain relieving lubricants also include pain absorbing material selected from the group defined as pain relieving lubricants.

In many cases, the pain relieving lubricants are in a container which is made of material selected from the group consisting of rolled laminated plastic and rolled laminated aluminum each having from one (1) to eight (8) layers of rolled laminated plastic or aluminum, plastic, polyurethane, and polyvinyl and combinations thereof, and metal including aluminum and precious metals including gold and silver. The plastic is selected from the group consisting of polypropylene, polyethylene, polyvinyl or combinations including one or more of these plastics. It is also within the spirit and scope of the present invention for the container to be made of metal materials, including aluminum and precious metal.

It is within the spirit and scope of the present invention for the container to be elongated with an oval or elliptical exterior shape such as a tube of toothpaste. It is also within the spirit and scope of the present invention for the container to be cylindrical in shape. In each shape of the respective containers, the container has a closed rear end, an exterior wall and a front wall, each having a interior surface surrounding an interior chamber retaining at least one of the pain relieving lubricants, and an open front end extending from an interior location of the front wall. In one embodiment, the open front end extends to a dispensing nozzle having an opening in a front wall and a threaded sidewall. The lubricant is dispensed by squeezing the sidewall and a lubricant is pushed out of the opening. Alternatively, a rollerball applicator extends from the interior surface out of the opening in the front wall.

An applicator is used to apply the pain relieving lubricants onto the skin at the area of the body which is a source of the pain, such as the lower back, knees, ankles, arms, elbows and neck. A preferred applicator for the present invention is a rollerball. Other dispensing applicators such as an open tube or nozzle are within the spirit and scope of the present invention.

In conventional containers for pain relieving lubricants, the applicator is covered with a removable cap. The present invention involves replacing the conventional cap with a cap that has a vibrator and a heating element. The cap also includes a selection switch associated with two spaced apart lights which shine through respective spaced apart openings in the cap. One light is blue to indicate no heat. The other light is red to indicate heat is generated from a heater within the cap. A vibrator is also within the cap.

The selection switch is used to turn on a vibrator and to turn on the heating element. Therefore, the present invention cap provides three separate sources to facilitate the skin absorbing at least one of the pain relieving lubricants. The cap is pressed against the area or moved around the area where the at least one of the pain relieving lubricants was applied. The three optional sources provided by the present invention are: (1) only heat, (2) only vibration; or (3) both vibration and heat.

It is therefore an object of the present invention to replace a conventional cap on a lubricant container with the present invention cap which contains the above described features to apply a source of heat and/or vibration to an area of pain on the body.

It is also an object of the present invention to include a child resistant locking assembly to reduce the ability of a child to remove the cap and gain access to at least one of the pain relieving lubricants within the container.

Further novel features and other objects of the present invention will become apparent from the following detailed description and discussion.

### BRIEF DESCRIPTION OF THE DRAWINGS

Referring particularly to the drawings for the purpose of illustration only and not limitation, there is illustrated:

FIG. 1 is a perspective view of a container in which at least one of the pain relieving lubricants is retained and the present invention cap placed onto the front of the container;

FIG. 2 is a side elevational view of the present invention cap;

FIG. 3 is an open view of the cap including a battery and wiring locations at one end to provide power to a heating element, a motor with a vibrator tip, a timer which by way of example is a 60 second timer, and an opening resistant or locking member to prevent unwanted opening of the cap;

FIG. 4 is a side view of the container with a transparent view of the front of the container and transparent view of the cap;

FIG. 5 is a perspective view illustrating the present invention cap with the front wall of the cap in place;

FIG. 6 is a perspective view illustrating the present invention cap with the front wall removed to illustrate an opening in the cap;

FIG. 7 is a side elevational view of the container with the cap removed and illustrating a pair of oppositely disposed tube standoffs on the front wall of the container;

FIG. 8 is a side perspective view of the container with the cap removed and illustrating a pair of oppositely disposed tube standoffs on the front wall of the container;

FIG. 9 illustrates an enlarged view of a tube standoff and its components;

FIG. 10 is a front view taken from lines 10-10 of FIG. 7, illustrating each tube standoff and its components;

FIG. 11 is a side view of the cap in partial cross-section to illustrate a portion of the locking assembly;

FIG. 12 is an exterior side view of the cap 20 of the present invention;

FIG. 13 is a cross-sectional view taken along line 13-13 of FIG. 12;

FIG. 14 is a cross-sectional view of the cap illustrating a pair of oppositely disposed cap locking teeth;

FIG. 15 is a cross-sectional view taken along line 15-15 of FIG. 14;

FIG. 16 is a bottom perspective view of the locking assembly on the cap;

FIG. 17 is a bottom cross-sectional view of the cap in the locked position;

FIG. 18 is an exploded view of the present invention cap and a side perspective view of an oval shaped container;

FIG. 19 is an exploded view of the present invention cap and a side perspective view of an oval shaped container;

FIG. 20 is a side perspective view of a container with the present invention cap in only the vibration mode;

FIG. 21 is a side perspective view of a container with the present invention cap in only the heating mode and hot and cold LED lights illuminated; and

FIG. 22 is a side perspective view of a container with the present invention cap in both the vibration mode and the heating mode, and hot and cold LED lights.

### DETAILED DESCRIPTION OF THE PREFERRED EMBODIMENTS

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention.

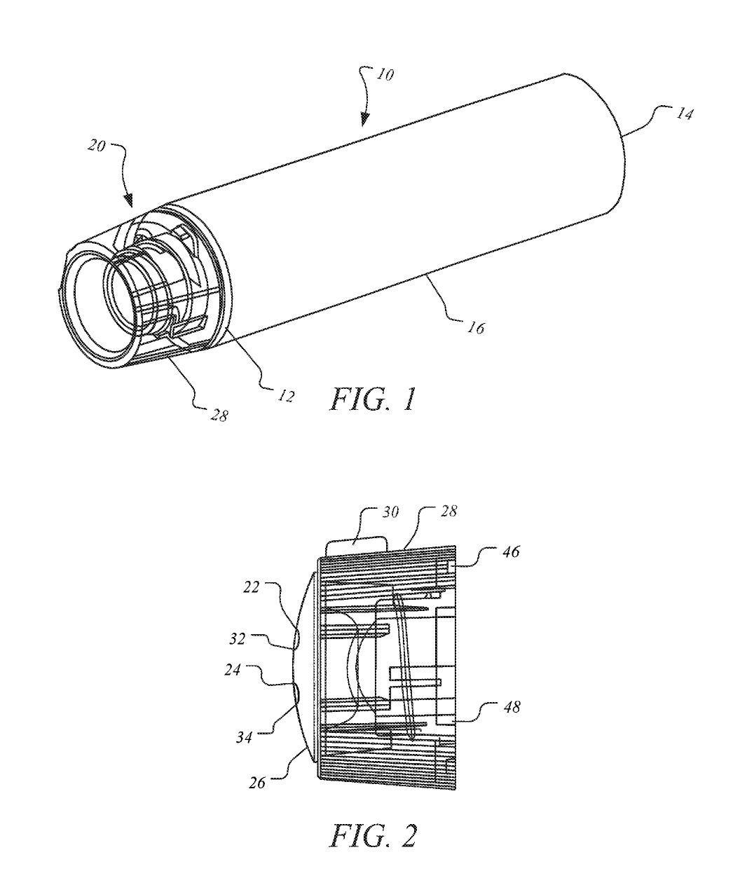

An embodiment of the present invention accessory cap is initially presented and used with a cylindrical shaped container. Referring to FIG. 1, there is illustrated a perspective view of a container in which at least one of the pain relieving lubricants is retained and the present invention cap placed onto the front of the container.

Further referring to FIG. 1, there is illustrated a perspective view of a container 10 in which at least one of the pain relieving lubricants is retained and the present invention cap 20 placed onto the front 12 of the container 10. The container 10 has a closed rear end 14 and a cylindrical sidewall 16. The rear end 14 and the cylindrical sidewall 16 surround an interior chamber to retain at least one of the pain relieving lubricants with the front end 12 open to which the present invention cap 20 is removably retained. As illustrated in FIG. 2, the present invention cap 20 has a cylindrical sidewall 28 and a front wall 26 which is either flat or convex shaped as illustrated in FIG. 2 with a first opening 22 through which a blue LED light 32 shines and a spaced apart second opening 24 through which a red LED light 34 shines. The color blue means cool or no heat is on and the color red means heat is on. A selection switch 30 which is a pushbutton is located adjacent front wall 26.

The container 10 is made of material selected from the group consisting of rolled laminated plastic and rolled laminated aluminum each having from one (1) to eight (8) layers of rolled laminated plastic or aluminum. The plastic is selected from the group consisting of polypropylene, polyethylene, polyvinyl or combinations including one or more of these plastics. It is also within the spirit and scope of the present invention for the container 10 to be made of other metal materials.

Referring to FIG. 2, there is illustrated a side elevational view of the present invention cap 20 with the blue LED light 32, the red LED light 34 and a power switch 30 which is preferably a pushbutton. There are also illustrated mating members 46 and 48 on a rear wall 44 of the cap 10.

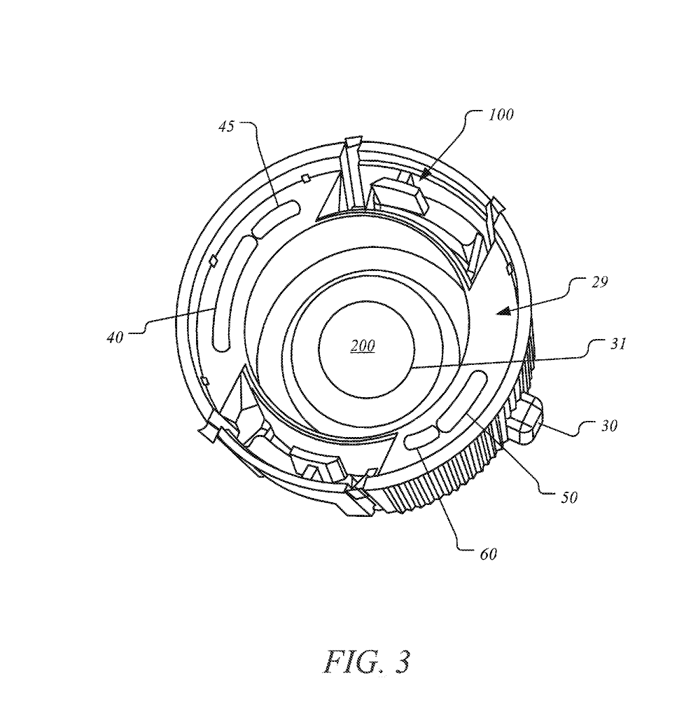

Referring to FIG. 3, there is illustrated an open interior view of the cap 20 including a battery and wiring location 40 at one end, also connected to a heating element 45. A motor with a vibrator tip 50 is at a different location within the cap 20. The battery and wiring 40 are also connected to the motor with a vibrator tip 50. The battery and wiring 40 are also connected to a timing member 60 which, as an example, is a 60 second timer for the indicator lights 32 and 34 and for both the heating element 45 and motor with vibrating tip 50. The cap 20 also includes a child resistant locking cap member 100 which is subsequently discussed in detail later in this patent application. The cap 20 has a sidewall 28 surrounding an interior chamber 29 in which the components 40, 45, 50 and 60 are retained. The cap 20 includes an interior opening 31 through which the rollerball applicator 200 is visible.

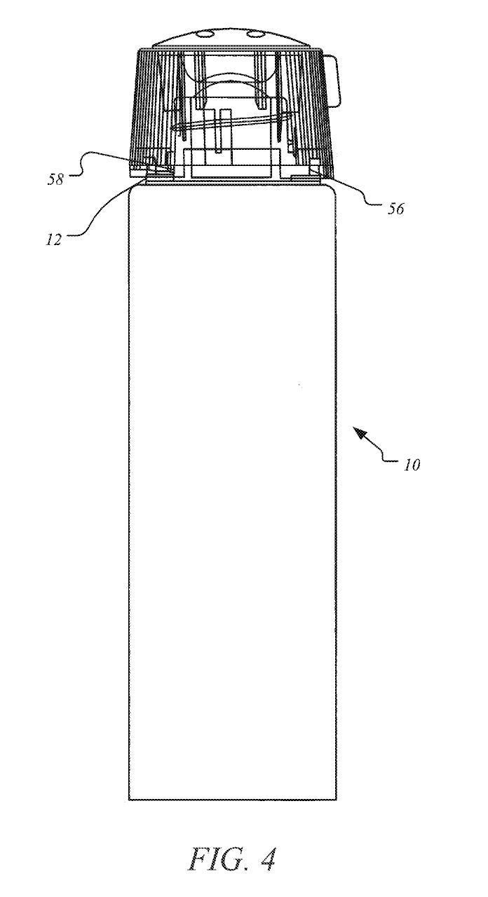

Referring to FIG. 4, there is illustrated a side view of the container 10 with a transparent view of the front 12 of the container 20 and transparent view of the cap 20. In one embodiment, extending out of the container front 12 there are a pair of spaced apart male mating members 56 and 58 which, by way of example, are pins respectively inserted into mating members 46 and 48 to removably retain the cap 20 onto the front 12 of the container 10. It is also within the spirit and scope of the present invention for the cap 20 to be removably retained onto the front 12 of the container 10 by a press fit or mating threads.

An optional feature of the present invention is a child-resistant locking assembly generally referred to as 100 in FIG. 3.

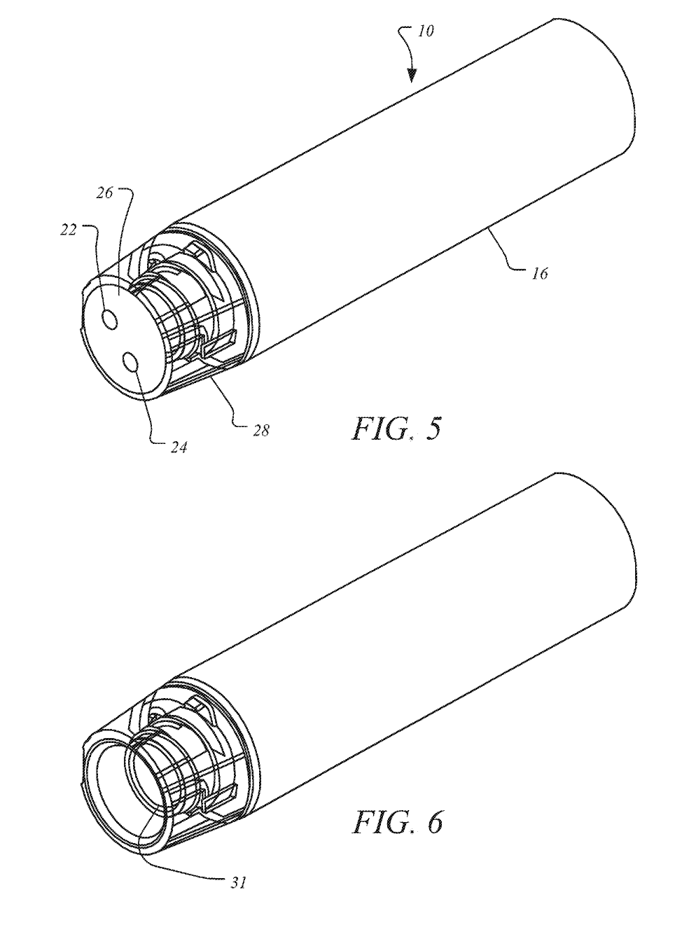

FIG. 5 is a perspective view illustrating an embodiment of the present invention cap 20 with the front wall in place and FIG. 6 is a perspective view with the front wall 26 removed to illustrate the opening 31 in cap 20.

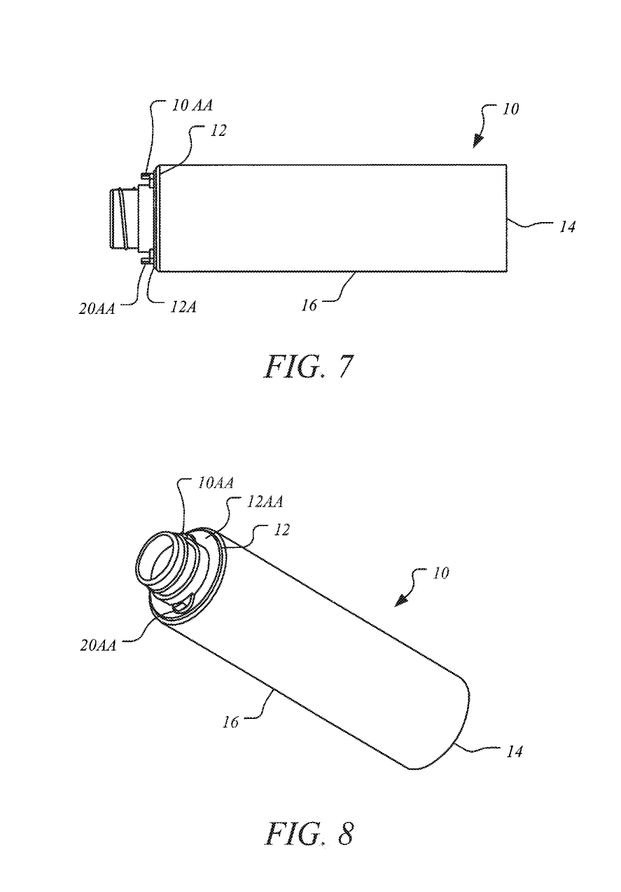

FIG. 7 is a side elevational view of the container 10 with the cap removed and illustrating a pair of oppositely disposed tube standoffs 10AA and 20AA on the front wall 12AA of the container 10.

FIG. 8 is a side perspective view of the container 10 with the cap removed and illustrating a pair of oppositely disposed tube standoffs 10AA and 20AA on the front wall 12AA to better illustrate the tube standoffs affixed to the front wall 12A on the container.

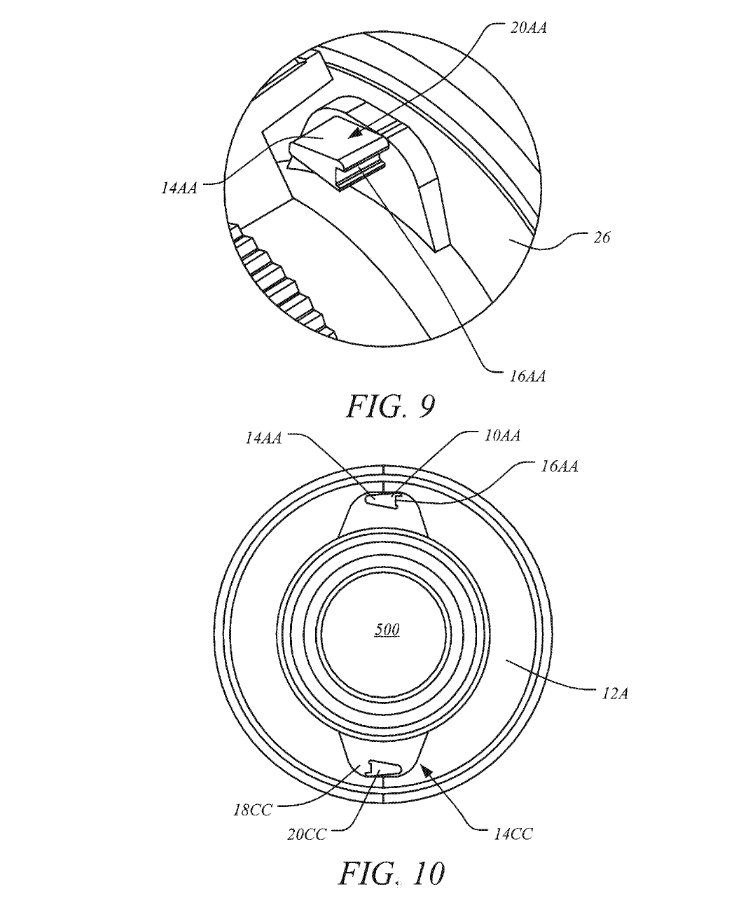

FIG. 9 illustrates an enlarged view of a tube standoff 10AA ands its components.

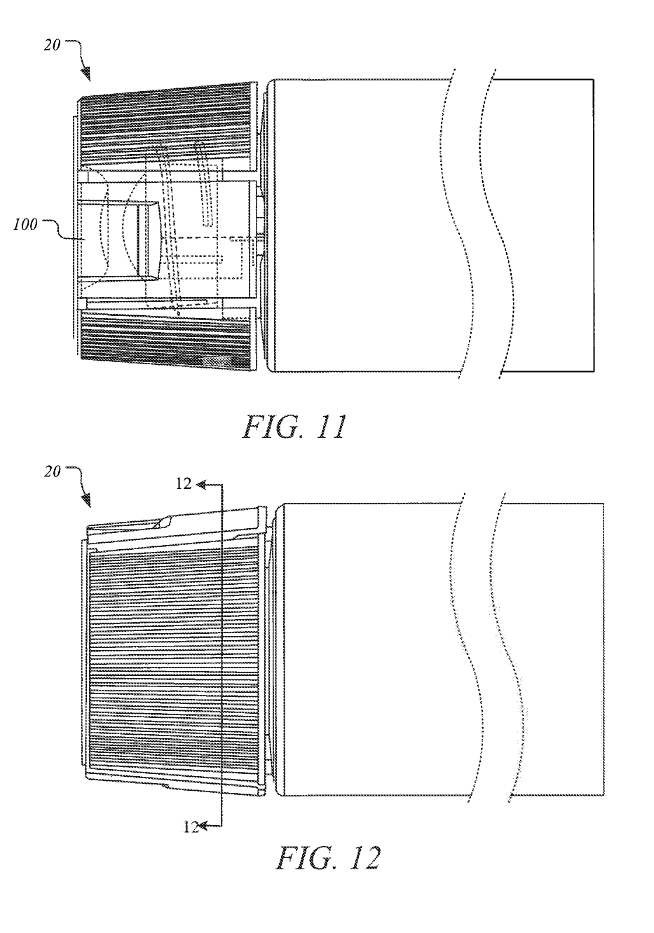

FIG. 10 is a front view taken from lines 10-10 of FIG. 7, illustrating tube standoff 10AA having a body 14AA and a cavity 16AA. Similarly, oppositely disposed tube standoff 20CC has a body 14CC with a cavity 16CC.

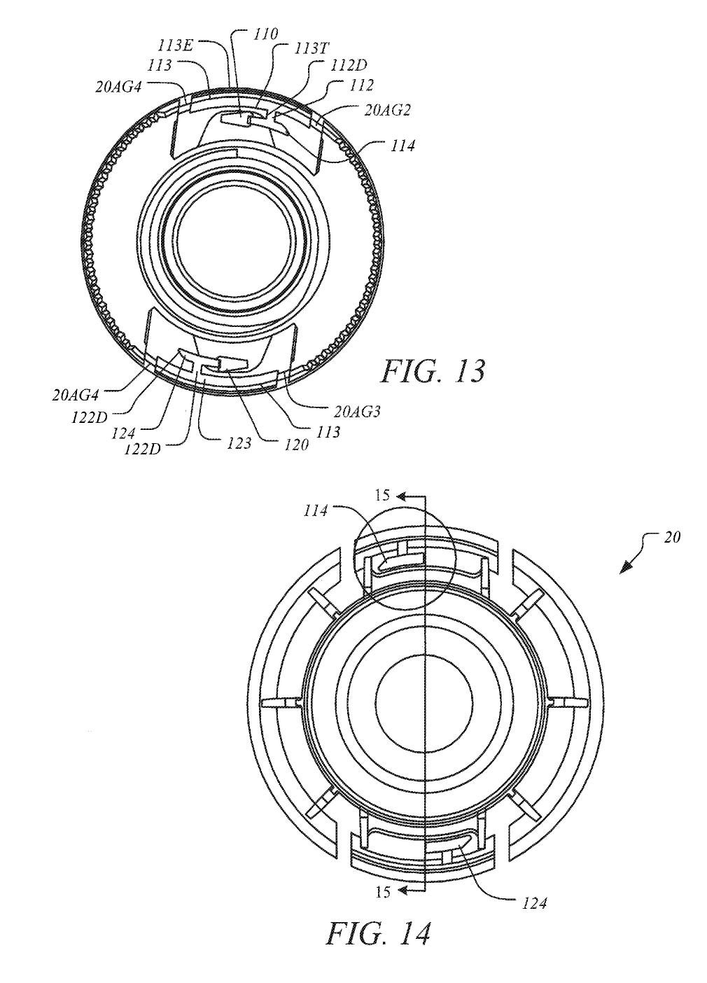

FIG. 11 is a side view of the cap 20 illustrated in partial cross-section to illustrate a portion of the locking assembly 100. FIG. 12 is an exterior side view of the cap 20 of the present invention. FIG. 13 is a cross-sectional view taken along line 13-13 of FIG. 12.

The cap 20 has a first locking member 110 having a transverse bar 112 extending from an interior wall section 113 toward the interior of the cap 20 and locking tooth 114 at the distal end 112D of transverse bar 112. A pair of gaps 20AG1 and 20AG2 are on either end of interior wall section 113. Similarly, cap 20 has a second locking member 120 having a transverse bar 122 extending from an interior wall section 123 extending toward the interior of cap 20 and a locking tooth 124 at the distal; end 122D of transverse bar 122. The pair of gaps 20AG3 and 20AG4 are on either end of interior wall section 123. Gaps 20AG1 and 20AG2 enable the cap 20 to be rotated. Similarly, gaps 20AG3 and 20AG4 enable the cap 20 to be rotated.

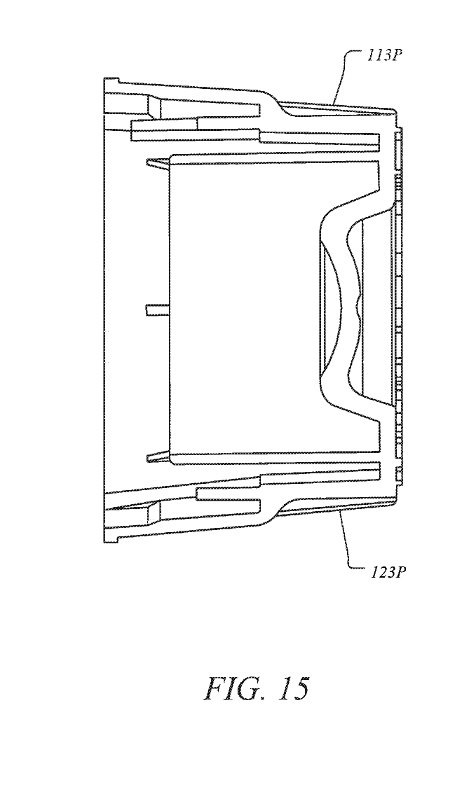

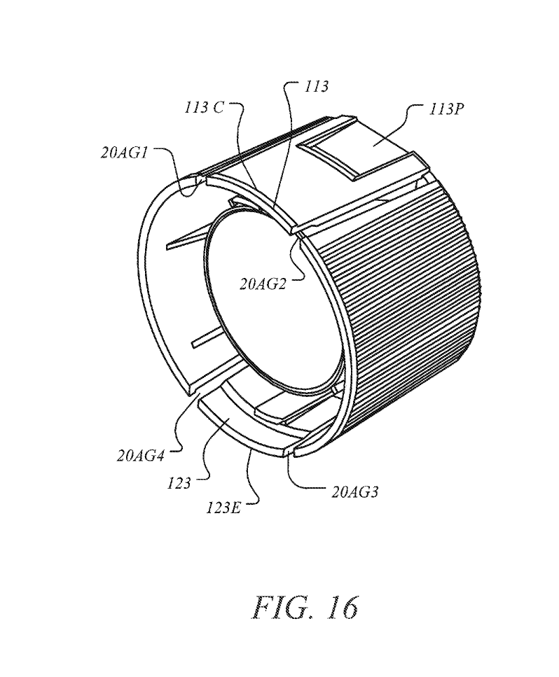

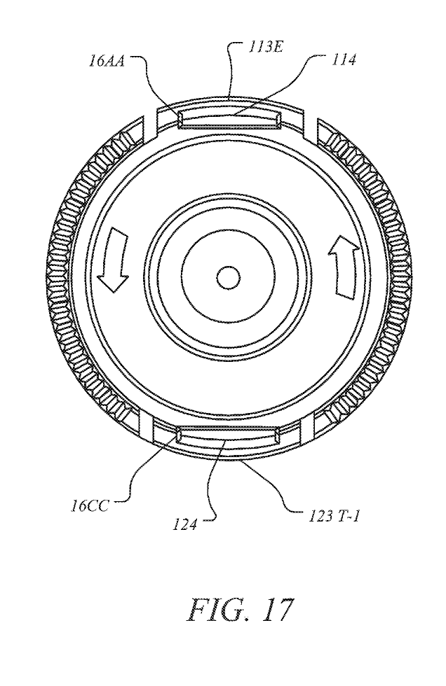

FIG. 14 is a cross-sectional view of the cap 20 illustrating a pair of oppositely disposed cap locking teeth 114 and 124. FIG. 15 is a cross-sectional view taken along line 15-15 of FIG. 14. FIG. 16 is a bottom perspective view of the locking 100 assembly on the cap 20. FIG. 17 is a bottom cross-sectional view of the cap 20 in the locked position.

In order to lock the cap 20, the cap 20 is pressed inwardly at the exterior wall location 133E corresponding to the location 133T of interior wall 113 where locking tooth 114 is located just in front of the cavity 16A of tube standoff 10AA and is concurrently pressed inwardly at the exterior wall location 133E corresponding to the location 133T of interior wall 133 where locking tooth 124 is located just in front of the cavity 16CC of tube standoff 10CC. Therefore, the tooth 114 is aligned with cavity 16AA of tube standoff 10AA and the tooth 124 is aligned with cavity 16CC of tube standoff 20AA. By rotating the cap 20 in one direction such as counterclockwise, tooth 114 engages and is inserted into cavity 16AA and the tooth 124 engages and is inserted into cavity 26CC to lock the cap 20 onto the cylindrical container 10. As illustrated in FIG. 17,

To unlock the cap 20, the cap 20 is pressed inwardly at the exterior wall location 113E corresponding to the location 113T-1 of interior wall 113 where locking tooth 114 is located inside of the cavity 16AA of tube standoff 10AA and is concurrently pressed inwardly at the exterior wall location 133E corresponding to the location 133T-1 of interior wall 133 where locking tooth 124 is inside of the cavity 16CC of tube standoff 10CC and then the cap 20 is rotated in an opposite direction (such as clockwise) to disengage tooth 114 from cavity 16AA and disengage tooth 124 from cavity 26CC and the pull cap 20A off of cylinder 10.

The container 10 has been illustrated as a cylindrical container. The present invention cap 10 with all of the features discussed above is also used with other shaped containers such as oval and elliptical with a dispensing nozzle or rollerball application.

The cap 20 has an exterior indentation member 113P and an oppositely disposed indentation member 123P. The indentation markers 113P and 123P serve as a guide as to where to inwardly press the cap 20 and rotate the cap into the locked or unlocked condition.

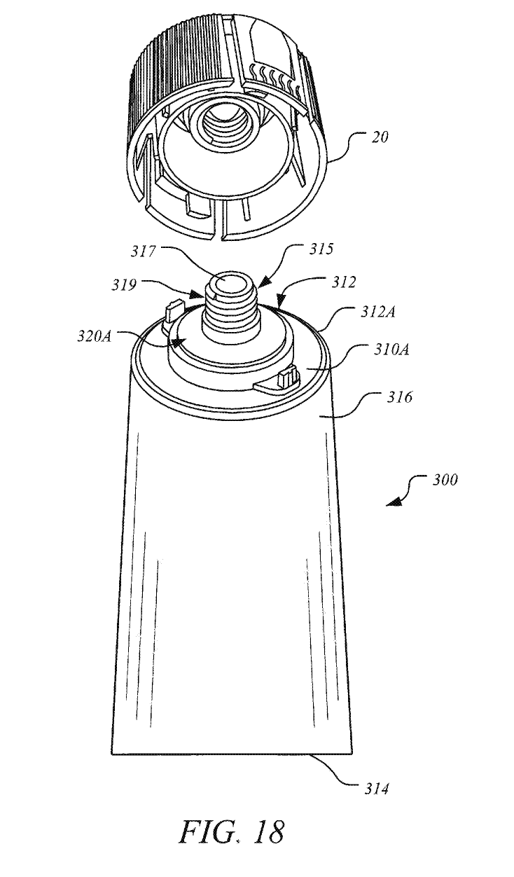

Referring to FIG. 18, there is illustrated an exploded view of the present invention cap and a side perspective view of an oval shaped container 300 in which at least one of the pain relieving lubricants is retained and the present invention cap 20 placed onto the front 312 of the oval shaped container 300. The container 300 has a closed rear end 314 and an oval shaped sidewall 316 which is flexible and squeezable to dispense the interior consents through an opening in a nozzle comparable to squeezing toothpaste out of a tube. A rear end 314 and the oval shaped I sidewall 316 surround an interior chamber to retain at least one of the pain relieving lubricants with the front end 312 having a front wall 312A with a central nozzle 315 having a central opening 317 through which the contents of the container is dispensed, The central nozzle 315 may have threads 319 on its cylindrical wall. The front wall 312A retains a pair of oppositely disposed standoffs 310AA and 320AA for the child-resistant locking mechanism as previously described.

The container 300 is also made of material selected from the group consisting of rolled laminated plastic and rolled laminated aluminum each having from one (1) to eight (8) layers of rolled laminated plastic or aluminum. The plastic is selected from the group consisting of polypropylene, polyethylene, polyvinyl or combinations including one or more of these plastics. It is also within the spirit and scope of the present invention for the container 10 to be made of other metal materials.

All of the features of the present invention cap 20 are the same as previously described.

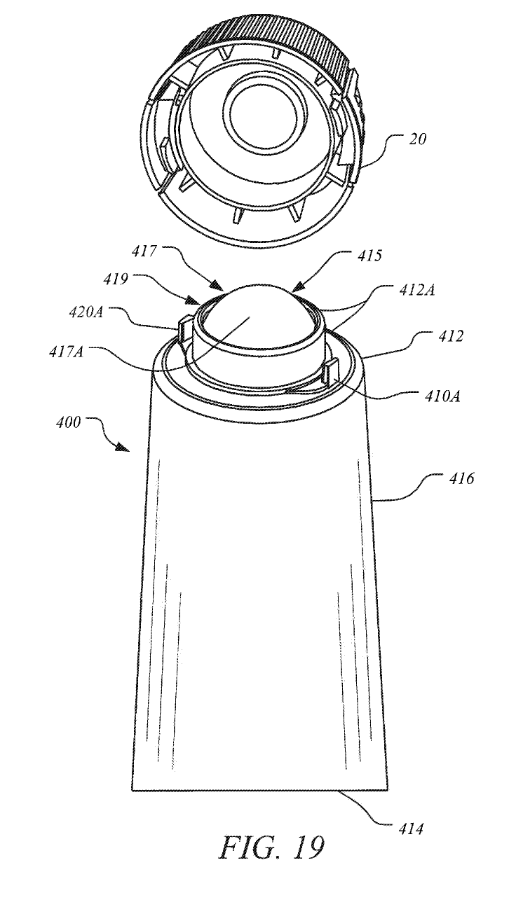

Referring to FIG. 19, there is illustrated an exploded view of the present invention cap 20 and a side perspective view of an elliptical shaped container 400 in which at least one of the pain relieving lubricants is retained and the present invention cap 20 placed onto the front 412 of the elliptical shaped container 400. The container 400 has a closed rear end 414 and an oval shaped sidewall 416 which is flexible and squeezable to dispense the interior consents through an opening in a nozzle comparable to squeezing toothpaste out of a tube. A rear end 414 and the oval shaped 1 sidewall 416 surround an interior chamber to retain at least one of the pain relieving lubricants with the front end 412 having a front wall 412A with a central nozzle 415 having a central opening 417 through which the contents of the container are dispensed. The central nozzle 415 may have threads 419 on its cylindrical wall. The front wall 412A retains a pair of oppositely disposed standoffs 410AA and 420AA for the child-resistant locking mechanism as previously described.

The container 410 is also made of material selected from the group consisting of rolled laminated plastic and rolled laminated aluminum each having from one (1) to eight (8) layers of rolled laminated plastic or aluminum. The plastic is selected from the group consisting of polypropylene, polyethylene, polyvinyl or combinations including one or more of these plastics. It is also within the spirit and scope of the present invention for the container 10 to be made of other metal materials.

All of the features of the present invention cap 20 are the same as previously described.

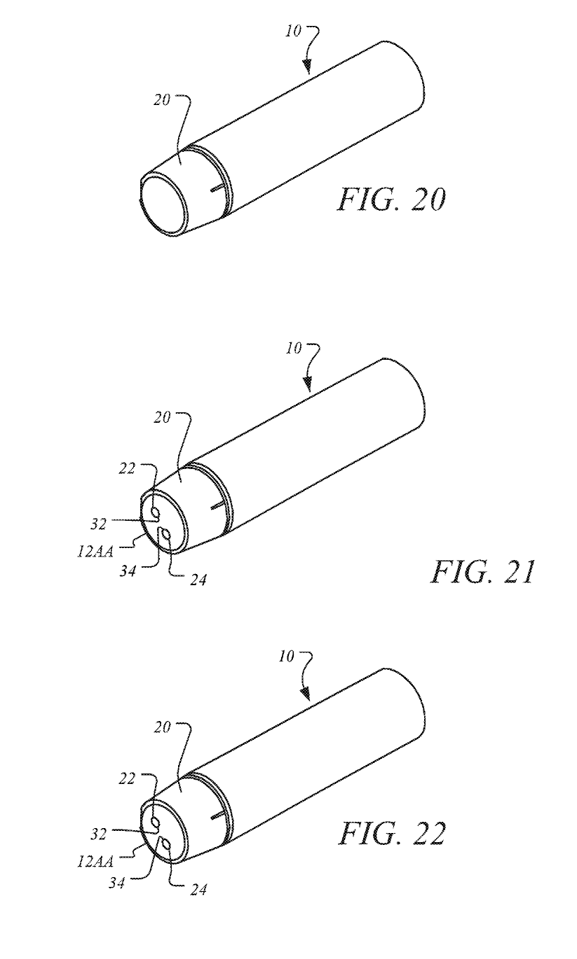

Referring to FIG. 20, there is illustrated a side perspective view of the a container 10 with the present invention cap 20 in only the vibration mode.

Referring to FIG. 21, there is illustrated a side perspective view of the container with the present invention cap 20 in only the heating mode and hot and cold LED lights.

Referring to FIG. 22, there is illustrated a side perspective view of the container 10 with the present invention cap 20 in both the vibration mode and the heating mode, and hot and cold LED lights.

Of course the present invention is not intended to be restricted to any particular form or arrangement, or any specific embodiment, or any specific use, disclosed herein, since the same may be modified in various particulars or relations without departing from the spirit or scope of the claimed invention hereinabove shown and described of which the apparatus or method shown is intended only for illustration and disclosure of an operative embodiment and not to show all of the various forms or modifications in which this invention might be embodied or operated.

What is claimed is:

1. An apparatus adapted for use with a container housing at least one pain relieving lubricant and an applicator, the apparatus comprising:
a. a cap removably affixed to a front of said container;
b. said cap having a sidewall and a front wall and a rear wall with an interior chamber including a battery and wiring connected to a heating element, a motor with a vibrator tip with the battery and wiring also connected to the motor with the vibrator tip;
c. said battery and wiring are also connected to a timing member, the timing member connected to hot and cold indicator lights, the timing member connected to said heating element and the timing member connected to said motor with a vibrating tip;
d. indicator lights shining through openings in as front wall of said cap;
e. said container including a closed rear end and a sidewall extending to a front wall having a centrally disposed member;
f. a first standoff affixed to said container front wall and an oppositely disposed second standoff affixed to said container front wall,
(i) the first standoff having a first body with a first cavity extending from a surface of the first body into the first body,
(iii) the second standoff having a second body with a second cavity extending from a surface of the second body into the second body;
g. said cap further including:
(i) a first locking member including a first transverse bar extending from a first interior wall section toward an interior of said cap and a first locking tooth at a distal end of the first transverse bar, first interior wall spacer cavities at both a first end and a second end of the first interior wall, the first locking tooth adapted to align with said first cavity,
(ii) said cap having a second locking member including a second transverse bar extending from a second interior wall section toward an interior of said cap and a second locking tooth at a di+++stal end of the second transverse bar, second interior wall spacer cavities at both a first end and a second end of the second interior wall, the second locking tooth adapted to align with said second cavity;
h. whereby, a simultaneous inward pressure on said cap at the locations of said first interior wall second and said second interior wall section and rotation of said cap enables said first tooth to engage said first cavity and said second tooth to engage said second cavity to lock said cap onto a front of said container, and removing said cap from said container requires finding the locations of said first interior wall and said second interior wall to cause a simultaneous inward force on said cap and anti-rotation of said cap to disengage said first tooth from said first cavity and said second tooth from said second cavity.

2. The apparatus in accordance with claim 1, further comprising: said indicator lights are two spaced apart LED lights which shine through respective spaced apart openings in said front wall of said cap.

3. The apparatus in accordance with claim 2, further comprising: one of said indicator lights emitting a blue color to indicate no heat, and a second of indicator lights emitting a red color red to indicate heat is generated from the cap.

4. The apparatus in accordance with claim 1, further comprising: only said heat element is activated.

5. The apparatus in accordance with claim 1, further comprising: only said motor with said vibrator tip is activate.

6. The apparatus in accordance with claim 1, further comprising: only said motor with said vibrator tip is activate.

7. The apparatus in accordance with claim 1, further comprising: both said heating element and said motor with said vibrator tip are activated.

8. The apparatus in accordance with claim 1, further comprising: said container is made of material selected from the group consisting of rolled laminated plastic and rolled laminated aluminum each having from one (1) to eight (8) layers of rolled laminated plastic or aluminum.

9. The apparatus in accordance with claim 8, further comprising: said rolled laminated plastic is selected from the group consisting of polypropylene, polyethylene, polyvinyl, and combinations including one or more of said polypropylene, polyethylene, polyvinyl.

10. The apparatus in accordance with claim 1, further comprising: said applicator is a rollerball.

11. The apparatus in accordance with claim 1, further comprising: said container sidewall is oval in shape.

12. The apparatus in accordance with claim 11, further comprising: said oval-shaped container sidewall is flexible and squeezable.

13. The apparatus in accordance with claim 1, further comprising: said container sidewall is elliptical in shape.

14. The apparatus in accordance with claim 13 further comprising: said elliptical-shaped container sidewall is flexible and squeezable.

15. The apparatus in accordance with claim 1, further comprising: said container sidewall is cylindrical in shape.

16. An apparatus adapted for use with a container housing at least one pain relieving lubricant, the apparatus comprising:
a. a cap removably affixed to a front of said container;
b. said cap having a sidewall and an interior chamber including a battery and wiring connected to a heating element, a motor with a vibrator tip with the battery and wiring also connected to the motor with the vibrator tip; and
c. indicator lights shining through openings in a front wall of said cap.

17. The apparatus in accordance with claim 16, further comprising: said battery and wiring are also connected to a timing member, the timing member connected to hot and cold indicator lights, the timing member connected to said heating element and the timing member connected to said motor with a vibrating tip.

18. The apparatus in accordance with claim 16, further comprising: said cap also including a locking member.

19. The apparatus in accordance with claim 18, further comprising: said locking member further includes:
a. a first standoff affixed to a front wall of aid container and an oppositely disposed second standoff affixed to said front wall of said container;
b. the first standoff having a first body with a first cavity extending from a surface of the first body into the first body;
c. the second standoff having a second body with a second cavity extending from a surface of the second body into the second body;
d. said cap having a first locking member including a first transverse bar extending from a first interior wall section toward an interior of said cap and a first locking tooth at a distal end of the first transverse bar, first interior wall spacer cavities at both a first end and a second end of the first interior wall, the first locking tooth adapted to align with said first cavity; and
e. said cap having a second locking member including a second transverse bar extending from a second interior wall section toward an interior of said cap and a second locking tooth at a distal end of the second transverse bar, second interior wall spacer cavities at both a first end and a second end of the second interior wall, the second locking tooth adapted to align with said second cavity;
f. whereby, a simultaneous inward pressure on said cap at the locations of said first interior wall second and said second interior wall section and rotation of said cap enables said first tooth to engage said first cavity and said second tooth to engage said second cavity to lock said cap onto a front of said container, and removing said cap from said container requires finding the locations of said first interior wall and said second interior wall to cause a simultaneous inward force on said cap and anti-rotation of said cap to disengage said first tooth from said first cavity and said second tooth from said second cavity.

20. The apparatus in accordance with claim 16, further comprising: said indicator lights are two spaced apart LED lights which shine through respective spaced apart openings in said front wall of said cap.

21. The apparatus in accordance with claim 20, further comprising: one of said indicator lights emitting a blue color to indicate no heat, and a second of indicator lights emitting a red color red to indicate heat is generated from the cap.

22. The apparatus in accordance with claim 16, further comprising: only said heat element is activated.

23. The apparatus in accordance with claim 16, further comprising: only said motor with said vibrator tip is activated.

24. The apparatus in accordance with claim 16, further comprising: only said motor with said vibrator tip is activated.

25. The apparatus in accordance with claim 16, further comprising: both said heating element and said motor with said vibrator tip are activated.

26. The apparatus in accordance with claim 16, further comprising: said container is made of material selected from the group consisting of rolled laminated plastic and rolled laminated aluminum each having from one (1) to eight (8) layers of rolled laminated plastic or aluminum.

27. The apparatus in accordance with claim 26, further comprising: said rolled laminated plastic is selected from the group consisting of polypropylene, polyethylene, polyvinyl, and combinations including one or more of said polypropylene, polyethylene, polyvinyl.

28. An apparatus adapted for use with a container housing at least one pain relieving lubricant, the apparatus comprising:
a. a cap removably affixed to a front of said container;
b. said cap having a sidewall and an interior chamber including a battery and wiring connected to a heating element, a motor with a vibrator tip with the battery and wiring also connected to the motor with the vibrator tip;
c. indicator lights shining through openings in a front wall of said cap;
d. said battery and wiring are also connected to a timing member, the timing member connected to hot and cold indicator lights, the timing member connected to said heating element and the timing member connected to said motor with a vibrating tip; and
e. said cap configured to operate selected from the group consisting of generating only vibration, generating only heat with at least a visible red light, and generating vibration combined with heat and at least one visible red light.

29. The apparatus in accordance with claim 28, further comprising: said cap also including a child-resistant locking member.
